(19) United States
(12) Patent Application Publication
(10) Pub. No.: US 2023/0083239 A1
(43) Pub. Date: Mar. 16, 2023

(54) Needle Having Multiple Dispensing Units

(71) Applicant: SNJ Patents, LLC, Palm Beach Gardens, FL (US)

(72) Inventor: Charles S. THEOFILOS, M.D., Palm Beach Gardens, FL (US)

(21) Appl. No.: 17/943,542

(22) Filed: Sep. 13, 2022

Related U.S. Application Data
(60) Provisional application No. 63/243,446, filed on Sep. 13, 2021.

(51) Int. Cl. A61M 5/14; A61M 5/162; A61M 5/315

(52) U.S. Cl. CPC A61M 5/1409; A61M 5/1626; A61M 5/31501

(57) ABSTRACT

A surgical instrument for delivering fluids or other agents or materials to an injection site, such as a surgical, treatment or tissue site. The surgical instrument comprises a multiple dispensing unit housing needle configured to house a plurality of fluid or materials dispensing units, or sub-needles, a needle cover sleeve, and an outer cannula. The surgical instrument is configured to provide fluid or materials to multiple areas of a surgical or treatment site during a single injection period, without having to remove the needle and re-insert to position it to inject fluid to another area of the surgical or treatment site.

### Cross-Reference To Related Applications

This application claims priority to U.S. Provisional Application No. 63/243,446, filed on Sep. 13, 2021, entitled “NEEDLE HAVING MULTIPLE DISPENSING UNITS,” which is hereby incorporated herein by reference in its entirety as an example.

### TECHNICAL FIELD

The invention relates generally to surgical equipment for injecting fluid or material; particularity, a surgical instrument for delivering fluid or material to a surgical or treatment site; and more particularly, to a surgical instrument having a needle with multiple fluid or material dispensing units for injecting fluid or material to multiple areas of a surgical or treatment site during a single injection period.

### BACKGROUND

Injection of fluids into a body is well known. For instance, substances may be injected into a patient in preparation for a surgical procedure, such as anesthesia injection for use in any surgical procedure requiring the patient to be “knocked out” during the actual procedure. Alternatively, substances, such as treatment agents, may be injected into a patient as part of the actual procedure. Such treatment agents may be anti-cancer drugs injected into a lesion or cancerous area. A needle with a single injection site is often used to dispense the treatment agent to the site. Since the single needle only dispenses the treatment agents to an area right at the injection site, numerous injections, which may require the needle to be removed and inserted numerous times, are generally required to fully treat a large treatment area.

Devices, such as those described in U.S. Pat. No. 6,217,554 and U.S. Pat. No. 7,087,040, using multiple needles to dispense fluids are known in the art. However, alternative surgical instruments capable of dispensing fluids using multiple dispensing units are needed.

### SUMMARY

Novel aspects of the present disclosure are directed to a surgical instrument for delivering fluid to multiple areas of an injection site. The surgical instrument can include a housing having a first end and a second end, and a plurality of flexible fluid dispensing units. Each of the plurality of flexible fluid dispensing units has a first end secured within the housing and a terminal end exposed at the second end of the housing, and at least one flexible fluid dispensing unit of the plurality of flexible fluid dispensing units has an initial configuration and a secondary configuration that differs from the initial configuration. The surgical instrument also includes a sleeve slidably engaged around the terminal ends of the plurality of flexible fluid dispensing units. The sleeve can be slidable from an initial position to a final position, which are relative to the terminal ends of the plurality of flexible fluid dispensing units. The initial position maintains the at least one flexible fluid dispensing unit in the initial configuration and the final position permits the at least one flexible fluid dispensing unit to transition to the secondary configuration. The at least one flexible fluid dispensing unit in the secondary configuration and the remaining flexible fluid dispensing units in the plurality of flexible fluid dispensing units can deliver the fluid from the first end of the housing to the multiple areas of the injection site when the sleeve is in the final position.

Novel aspects of the present disclosure are also directed to a method of operating a surgical instrument for delivering fluid to multiple areas of an injection site. The method includes positioning the surgical instrument at the injection site to deliver the fluid and then advancing a housing towards the injection site. The housing includes a first end and a second end, and a plurality of flexible fluid dispensing units secured within the housing with terminal ends of the plurality of flexible fluid dispensing units exposed at the second end of the housing.

The method also includes sliding a sleeve from an initial position to a final position relative the terminal ends of the plurality of flexible fluid dispensing units. The initial position maintains at least one flexible fluid dispensing unit of the plurality of flexible dispensing units in an initial configuration, and the final position permits the at least one flexible fluid dispensing unit to transition to a secondary configuration that differs from the initial configuration. Fluid is then delivered from the first end of the housing and though the terminal ends of at least some of the plurality of fluid dispensing units to the multiple areas of the injection site.

### BRIEF DESCRIPTION OF THE DRAWINGS

FIG. 1 is a perspective view of an illustrative embodiment of a needle having multiple dispensing units;

FIG. 2 is an exploded view of the needle having multiple dispensing units;

FIG. 3 is a perspective view of the multiple dispensing unit housing structure;

FIG. 4 is a cross-sectional view taken along lines A-A in FIG. 1;

FIG. 5 is an enlarged view of the area shown in Circle D of FIG. 2;

FIG. 6 is an enlarged view of the area shown in Circle C of FIG. 2;

FIG. 7 illustrates an embodiment of the fluid dispensing units or sub-needles;

FIG. 8 illustrates an embodiment of the needle having multiple dispensing units having fluid dispensing unit(s) of the same length and same angle orientation;

FIG. 9 illustrates an embodiment of the needle having multiple dispensing units having fluid dispensing unit(s) of different lengths;

FIG. 10 illustrates an embodiment of the needle having multiple dispensing units having fluid dispensing unit(s) of the same angle orientations;

FIG. 11 illustrates an embodiment of the needle having multiple dispensing units having fluid dispensing unit(s) of different angle orientations;

FIG. 12 illustrates an embodiment of the needle having multiple dispensing units having fluid dispensing unit(s) arranged for a 360-degree dispersal of fluids;

FIG. 13 illustrates an embodiment of the needle having multiple dispensing units configured to dispense fluids simultaneously;

FIG. 14 illustrates an embodiment of the needle having multiple dispensing units configured to dispense (or not dispense) fluids independently;

FIG. 15 is a perspective view of an illustrative embodiment of a needle having multiple dispensing units;

FIG. 16 is a perspective view of the needle having multiple dispensing units illustrated in FIG. 15, shown with the multiple dispensing units;

FIG. 17 is an exploded view of the needle having multiple dispensing units illustrated in FIG. 15;

FIG. 18 is a planar view of the needle having multiple dispensing units illustrated in FIG. 15, shown with the multiple dispensing units in a covered position;

FIG. 19 is a planar view of the needle having multiple dispensing units illustrated in FIG. 15, shown with the multiple dispensing units in a non-covered position;

FIG. 20 is a cross-sectional view taken along lines B-B in FIG. 18;

FIG. 21 is a cross-sectional view taken along lines C-C in FIG. 18;

FIG. 22 illustrates the needle having multiple dispensing units with luer lock needle(s);

FIG. 23 is a plan view of the needle having multiple dispensing units with luer lock needle(s) shown in FIG. 22; and

FIG. 24 is a cross-sectional view taken along lines D-D in FIG. 23.

### DETAILED DESCRIPTION

While the present invention is susceptible of embodiment in various forms, there is shown in the drawings and will hereinafter be described a presently preferred, albeit not limiting, embodiment with the understanding that the present disclosure is to be considered an exemplification of the present invention and is not intended to limit the invention to the specific embodiments illustrated.

Embodiments of the invention describe a surgical instrument useful for delivery of fluids or other agents or materials to an injection site, such as a surgical site, a treatment site, or a tissue site. While the surgical, treatment, or tissue site may be described with reference to spinal surgery, such as the vertebral body, the surgical instrument in accordance with embodiments of the invention are not limited to use in spinal surgery and may be used in other surgeries or for other parts of a mammalian, such as a human, body. The surgical instrument is configured to provide fluid (or materials) to multiple areas of a surgical or treatment site during a single injection period, i.e. without having to remove the needle and re-insert to position it to inject fluid to another area of the injection site. The surgical instrument is an improvement over other needles currently in use in that the surgical instrument provides a device that delivers fluid or material to the surgical, treatment, or tissue site in a more controlled and precise manner.

Accordingly, it is an objective of the invention to provide an improved surgical instrument for dispensing fluid or material.

It is a further objective of the invention to provide a surgical instrument configured to provide fluid or material to multiple areas of an injection site without having to remove the needle and re-insert to position it to inject fluid or material to another area of the injection site.

It is a further objective of the invention to provide a surgical instrument having multiple fluid or material dispensing units for dispensing fluid to a surgical site.

It is yet another objective of the invention to provide a surgical instrument having multiple fluid or material dispensing units for dispensing fluid to a tissue site.

It is a still further objective of the invention to provide a surgical instrument having multiple fluid or material dispensing units for dispensing fluid to a surgical site.

It is a further objective of the invention to provide a surgical instrument having multiple fluid or material dispensing units, each configured to have the same length.

It is yet another objective of the invention to provide a surgical instrument having multiple fluid or material dispensing units, each configured to have the same angle of orientation.

It is a still further objective of the invention to provide a surgical instrument having multiple fluid or material dispensing units, each configured to have different lengths.

It is a further objective of the invention to provide a surgical instrument having multiple fluid or material dispensing units, each configured to have different angles of orientation.

It is yet another objective of the invention to provide a surgical instrument having multiple fluid or material dispensing units configured for up to 360-degree dispersal of fluids or materials.

It is a still further objective of the invention to provide a surgical instrument having multiple fluid or material dispensing units, each configured to dispense fluids simultaneously.

It is a further objective of the invention to provide a surgical instrument having multiple fluid or material dispensing units, each configured to dispense (or not dispense) fluids or material separately and/or independently.

Other objectives and advantages of this invention will become apparent from the following description taken in conjunction with any accompanying drawings wherein are set forth, by way of illustration and example, certain embodiments of this invention. Any drawings contained herein constitute a part of this specification, include exemplary embodiments of the present invention, and illustrate various objects and features thereof.

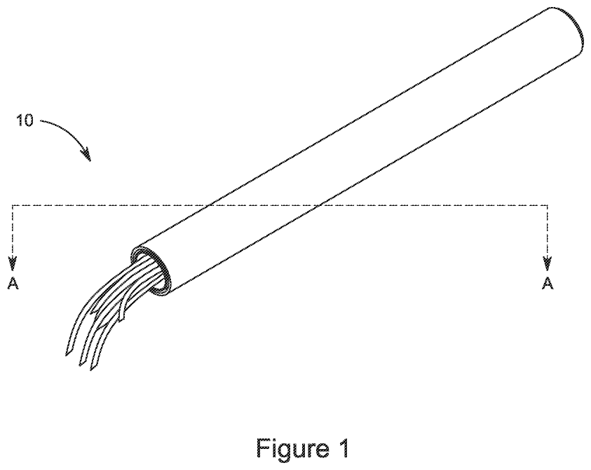

Referring to FIGS. 1-4, an illustrative embodiment of a surgical instrument having multiple delivery units, referred to generally as a needle having multiple dispensing units 10, is shown. The needle having multiple fluid dispensing units 10 comprises a multiple dispensing unit housing needle 12 configured to hold or house a plurality of fluid or material dispensing units, also referred to as sub-needles, 14, a needle cover sleeve 16, and an outer cannula 18. Each fluid dispensing unit may be referred to individually as fluid dispensing unit 14A, fluid dispensing unit 14B, fluid dispensing unit 14C, fluid dispensing unit 14D, fluid dispensing unit 14E, fluid dispensing unit 14F, and fluid dispensing unit 14G.

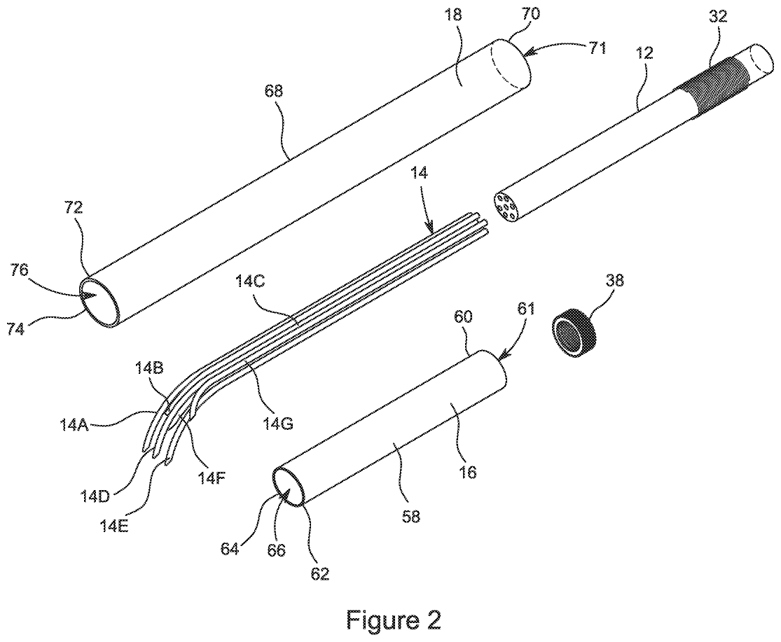

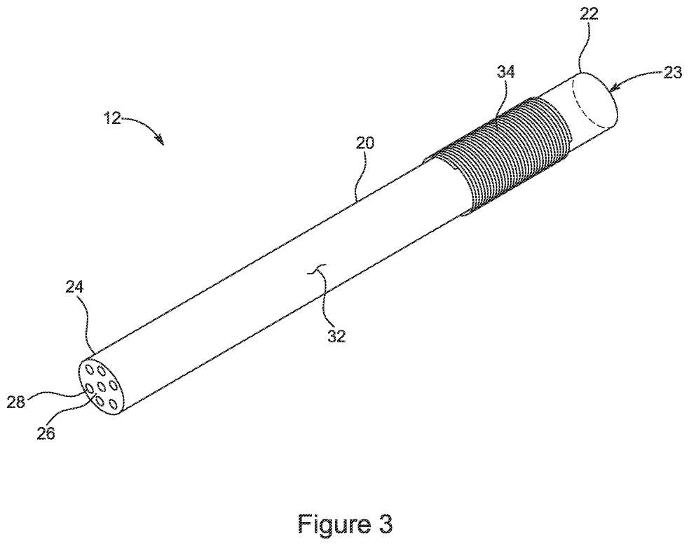

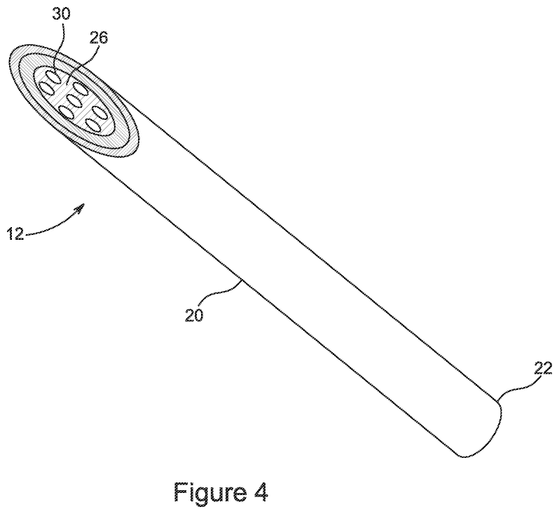

Referring to FIG. 3, the multiple dispensing unit housing structure 12 is shown as an elongated body 20 having a first end 22 (with opening 23) and a second, opposing end 24. An inner lumen 26 runs the entire length of the elongated body 20, from the first end 22 to the second, opposing end 24. The inner lumen 26 is shown having a solid interior having a plurality of inner lumen openings 28. The plurality of inner lumen openings 28 are sized and shaped to receive and hold therein at least a portion of the fluid dispensing units 14. The inner lumen openings 28 may include internal fluid passageways 30, see FIG. 4, that extend to the first end 22 or any distance between the second, opposing end 24 and the first end 22. Towards the first end 22, the outer surface 32 may include threading 33, see FIGS. 2 and 5. The threading 33 corresponds to and engages with the inner threading 36 of the stop ring 38, see FIG. 6.

The stop ring 38 is designed to prevent the multiple dispensing unit housing structure 12 from sliding completely within the outer cannula 18 and/or the needle cover sleeve 16. Adjusting the stop ring 38 up/down, the threading 32 provides depth adjustment, i.e. control the depth of insertion, and ultimately advancing or retracting the sub-needles 14. Such depth provides for controlling the sub-needles 14 depth placement, i.e. deeper or shallower extension. The outer surface 41 of the stop ring 38 comprises a plurality of surface configurations 42, and configured to assist in advancing or retracting the sub-needles 14. An adapter may be added at the first end 22 to attach a syringe, leur lock, slip tip, IV bag, etc, to allow for fluid to be delivered to the sub-needles 14.

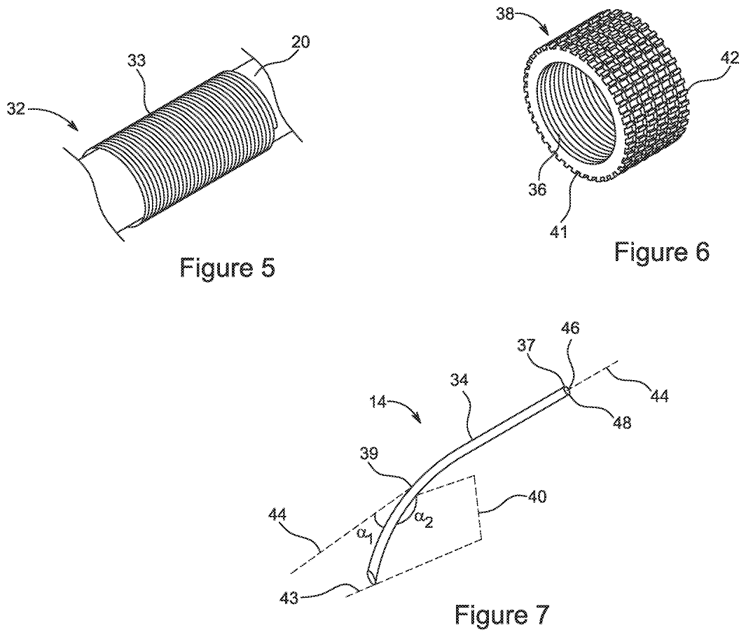

The plurality of fluid dispensing units 14 are configured to allow passage of fluid there through. Accordingly, each of the fluid dispensing units, fluid dispensing unit 14A, fluid dispensing unit 14B, fluid dispensing unit 14C, fluid dispensing unit 14D, fluid dispensing unit 14E, fluid dispensing unit 14F, and fluid dispensing unit 14G, may comprise a main body 34 having a first end 37 and a second end 39, see FIG. 7. Preferably, the length of the main body 34 between the first end 37 and the second end 39 toward is positioned within the inner lumen openings 28, and may include internal fluid passageways 30. Extending from the second end 39, the fluid dispensing units 14 comprise a pre-bent, flexible fluid dispensing portion 40. The pre-bent, flexible fluid dispensing portion 40 may comprise a terminal end 43 having an opening to allow fluid to flow out from the fluid dispensing units 14. Accordingly, the length of the sub-needles 14, from first end 37 to the terminal end 43 is positioned within the inner lumen openings 28.

The pre-bent, flexible fluid dispensing portion 40 is configured to traverse or move from a first non-bent or straight position, i.e. resting position, to a bent position. Accordingly, the pre-bent, flexible fluid dispensing portion 40 is designed to have a predetermined angle, a, relative to a longitudinal axis 44. The predetermined angle a may be any angle between 0 degrees and 180 degrees. While the pre-bent, flexible fluid dispensing portion 40 is shown oriented below the longitudinal axis 44, the predetermined angle, a, may be formed above the longitudinal axis 44 as well. When a force is applied to the pre-bent, flexible fluid dispensing portion 40, the pre-bent, flexible fluid dispensing portion 40 will assume a straight/linear orientation or move towards a straight/linear orientation, i.e. no longer be bent at the predetermined bent angle. Once the force is removed, the pre-bent, flexible fluid dispensing portion 40 assumes its original, pre-bent orientation. The first end 37 of the main body 34 may include an opening 46, exposing an internal portion or lumen 48. The internal portion or lumen 48 is sized and shaped to allow fluid to flow toward the pre-bent, flexible fluid dispensing portion 40. The fluid dispensing unit 14 is preferably made of Nitinol, but may be made of any material to accomplish its functions.

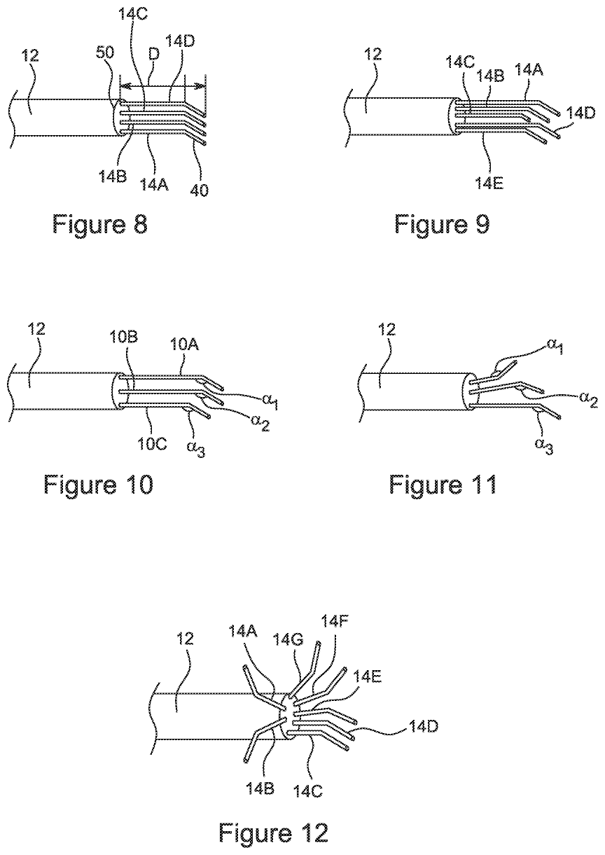

Based on the arrangement or orientation of the fluid dispensing unit(s) 14, the needle having multiple dispensing units 10 provides dispersion of liquid in multiple patterns. For example, the needle having multiple dispensing units 10 may include all fluid dispensing unit(s) 14 having the same length and same angle orientation. Referring to FIG. 8, fluid dispensing unit 14A, fluid dispensing unit 14B, fluid dispensing unit 14C, and fluid dispensing unit 14D all have the same length (distance D, from the end 50 of the main multiple dispensing unit housing needle 12 to the start of the fluid dispensing portion 40 or terminal point 43 of the fluid dispensing unit 14).

The needle having multiple dispensing units 10 may include fluid dispensing units 14 having different lengths, with the same angle orientations or differing angle orientations. Referring to FIG. 9, fluid dispensing unit 14A, fluid dispensing unit 14B, fluid dispensing unit 14C, fluid dispensing unit 14D, and fluid dispensing unit 14E have different lengths. As shown in the Figure, fluid dispensing unit 14C has the smallest length. Fluid dispensing units 14A and 14D have the longest lengths. Fluid dispensing units 14B and 14E have different lengths, which fall between the lengths fluid dispensing unit 14C and fluid dispensing units 14A, 14D. Arranging the fluid dispensing units at different lengths provides for dispensing fluids to a site at different depths.

The needle having multiple dispensing units 10 may include fluid dispensing units 14 having the same predetermined angle, a. Referring to FIG. 10, fluid dispensing unit 14A, fluid dispensing unit 14B, and fluid dispensing unit 14C are shown having the same predetermined angle, a, such as all being 30 degrees, all being 45 degrees, all being 90 degrees. The needle having multiple dispensing units 10 may include fluid dispensing units 14 having different predetermined angles, a. Referring to FIG. 11, fluid dispensing unit 14A (α1), fluid dispensing unit 14B (α2), and fluid dispensing unit 14C (α3) are shown having different predetermined angles.

The needle having multiple dispensing units 10 may include fluid dispensing units 14 arranged or orientated to provide a particular fluid dispensing pattern. Referring to FIG. 12, the needle having multiple dispensing units 10 is shown having the fluid dispensing units 14 (14A-14F) arranged to provide a 360-degree spray pattern. The needle having multiple dispensing units 10 may be configured to provide a fluid spray pattern of between zero degrees and 360 degrees.

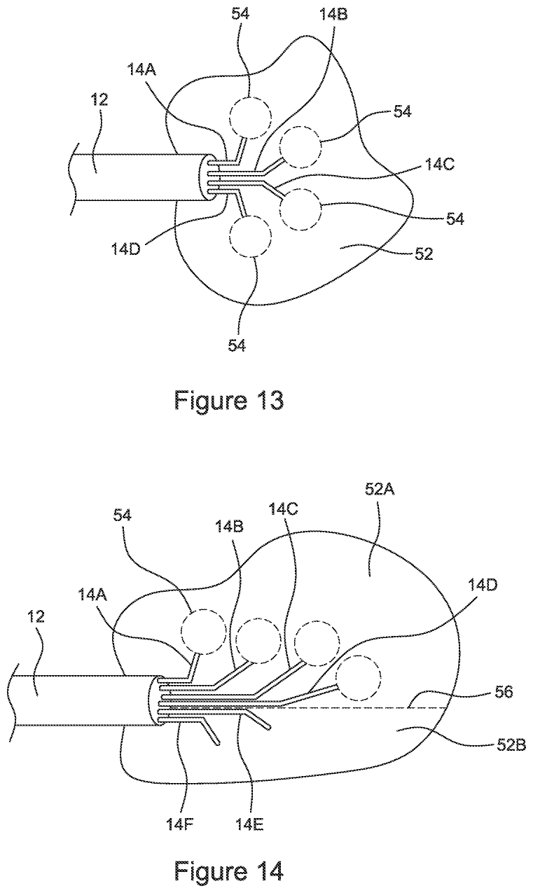

In use, the needle having multiple dispensing units 10 may be configured to allow for each of the fluid dispensing units 14 to dispense fluids simultaneously. Alternatively, the needle having multiple dispensing units 10 may be configured to allow for each of the fluid dispensing units 14 to dispense fluids at different times, or not at all. Referring to FIG. 13, the needle having multiple dispensing units 10 is shown inserted into a surgical or treatment site 52, such as a vertebral body. As depicted in FIG. 13, each fluid dispensing unit 14A-D is dispensing fluid, see dotted line circles 54. As depicted in FIG. 14, each fluid dispensing unit 14A-D is dispensing fluid, see dotted line circles 54, to a portion of the surgical or treatment site 52. Fluid dispensing units 14E and 14F are shown not dispensing fluid. In this manner, the user is capable of dispense fluid to one portion of a site, section 52A above broken line 56, but not another site, section 52B below dotted line 56.

Referring back to FIG. 2, the needle cover sleeve 16 is shown as a tubular body 58 having a first end 60 and an opposing, second end 62. The first end 60 has an opening 61. The second end 62 has an opening 64, exposing an open (hollow or partially hollow) interior 66. The needle cover sleeve 16 is sized and shaped to cover and slide over the fluid dispensing units 14 and/or the multiple dispensing unit housing structure 12. The outer cannula 18 is shown as a generally tubular shaped body 68 having a first end 70 and an opposing second end 72. The first end 70 has an opening 71. The second end 72 has an opening 74, exposing an open (hollow or partially hollow) interior 76. The outer cannula 18 is sized and shaped to allow the multiple dispensing unit housing needle 12 to fit and move within. Individual dispensing units, such as syringes (not shown), may also be secured to each of the multiple fluid dispensing units 14 as a source of fluid dispensing. The outer cannula 18 may be configured, such as through inclusion of a stop, lip, or inwardly tapering, at the second end to prevent the needle cover sleeve tubular body 58 from extending out the past the second end 72.

In use, the user, such as a surgeon, would place the outer cannula 18 at a surgical or treatment site in need of injection of a fluid thereto. The outer cannula 18 may be directed to the site using a stylet (not shown) similar to Jamshidi biopsy needles. Once at the correct site, the multiple dispensing unit housing needle 12 with multiple fluid dispensing units 14 may be inserted into the outer cannula 18. Use of the needle cover sleeve 16 maintains the multiple fluid dispensing units 14 in the non-bent state. Once at the location of the surgical or treatment site, as the dispensing unit housing needle 12 is forced in, the outer cannula 18 holds back the needle cover sleeve 16, exposing the pre-bent sub-needles 14. In this position, the sub-needles 14 traverse from the non-bent position to the bent shape orientation. In this position, fluid may be dispersed to the surgical or treatment site.

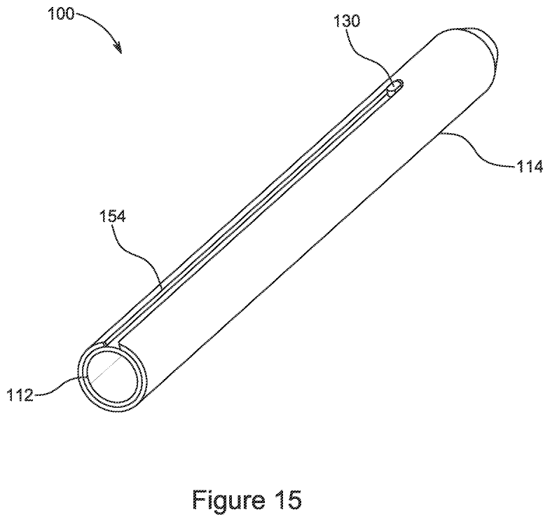

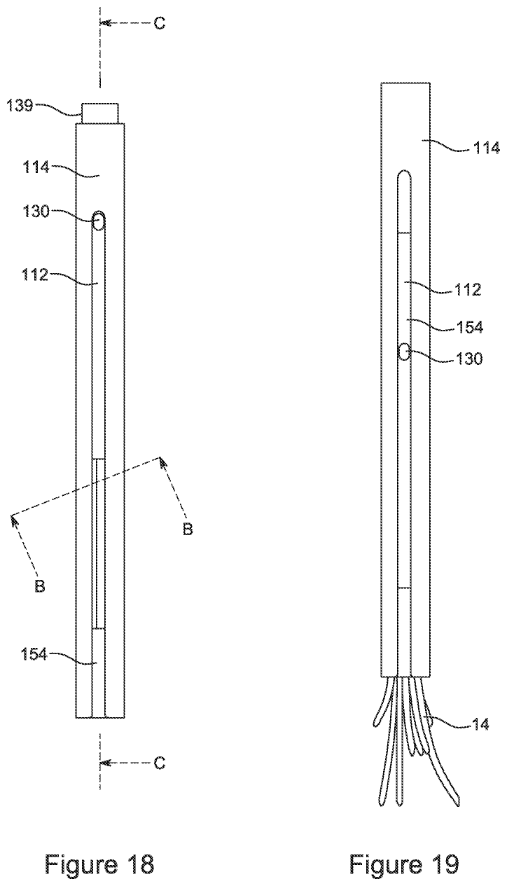

Referring to FIGS. 15-21, an alternative embodiment of the needle having multiple dispensing units, referred to as the needle having multiple dispensing units 100, is shown. The needle having multiple dispensing units 100 has the same or similar structural components as that described for the needle having multiple dispensing units 10. Where the structural components are the same, the same reference numbers are used for the needle having multiple dispensing units 10 as the needle having multiple dispensing units 100. The needle having multiple fluid dispensing units 100 comprises a multiple dispensing unit housing needle 112 configured to hold or house the plurality of fluid dispensing units, also referred to as sub-needles, 14, a needle cover sleeve 16, and an outer cannula 114.

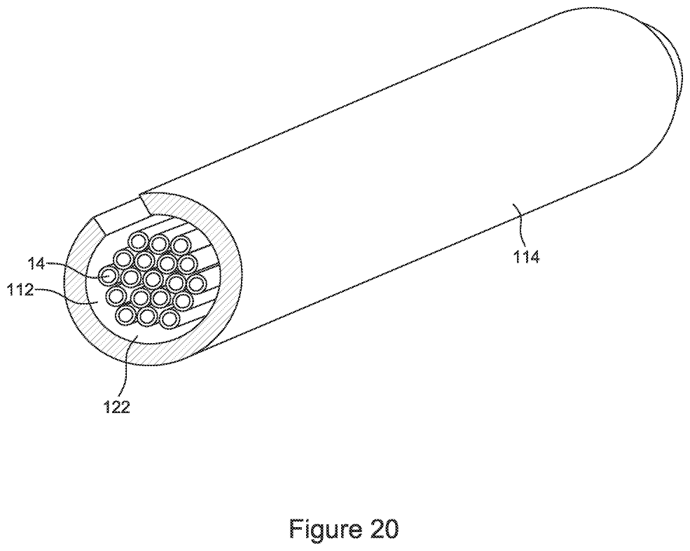

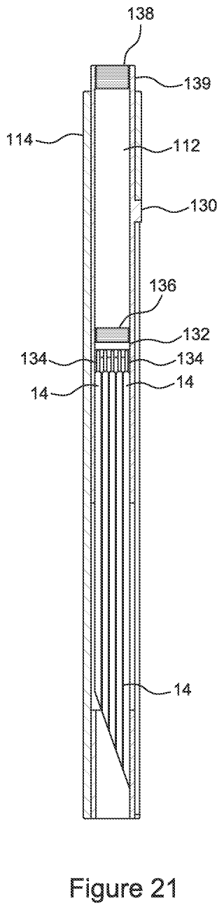

The multiple dispensing unit housing needle 112 is shown as an elongated body 116 having a first end 118 and a second, opposing end 120. An inner lumen 122, see FIG. 20, runs the entire length of the elongated body 120, from the first end 118 to the second, opposing end 120. The inner lumen 122 is shown being hollow. Alternatively, the inner lumen 122 may be configured having a solid interior having a plurality of inner lumen openings and internal fluid passageways, as described previously. The first end 118 includes an opening 124. The second, opposing end 120 includes opening 126 of sufficient size to allow the plurality of fluid dispensing units to fit within or extend out from. The outer surface 128 of the multiple dispensing unit housing needle elongated body 116 includes a stop member 130, illustrated herein as a shaped protuberance, having sufficient size and profile to engage with at least a portion of the outer cannula 114. A needle cap 132 contains one or more fluid dispensing unit engaging members 134, illustrated herein as tubular shaped bodies. Each individual fluid dispensing unit engaging member is secured or engages with each individual fluid dispensing unit 14, see FIG. 21, to allow fluid to flow through the needle cap 132 and into each individual fluid dispensing unit 14. The needle cap 132 may also include a threaded junction 136 to provide attachment to a fluid dispensing device, such as a syringe, IV bag or other attachment devices, such as a luer lock connector, slip tip, etc. An opening 138 with threading 139 may be contiguous with the threaded junction 136 to prevent fluid from leaking out.

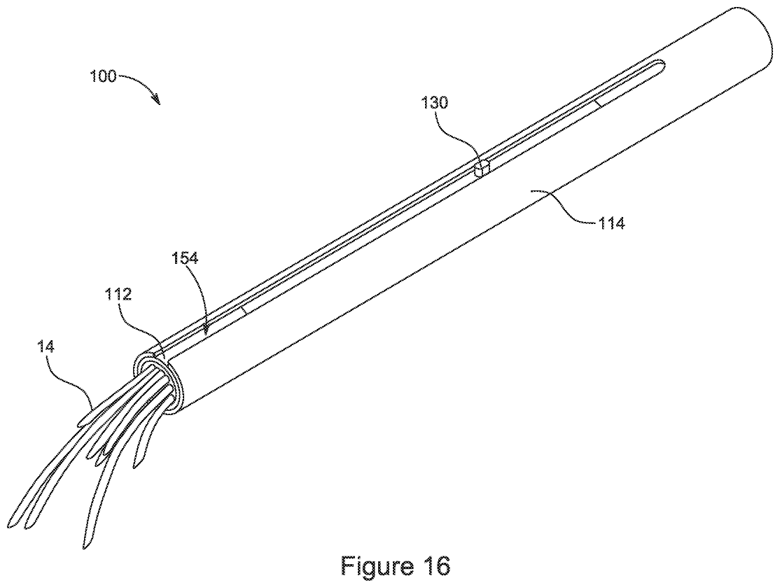

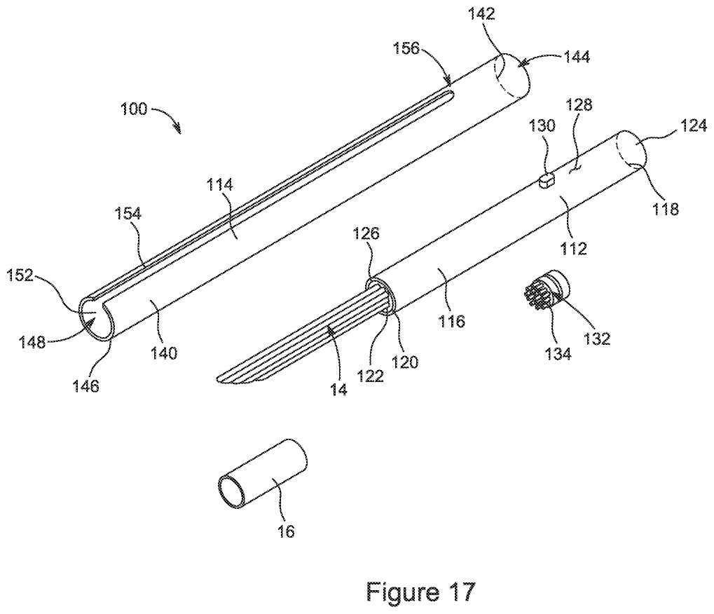

The outer cannula 114 is shown as a generally tubular shaped body 140, see FIG. 17, having a first end 142, with an opening 144, and an opposing second end 146, with an opening 148. An open (hollow or partially hollow) interior 152 runs the entire length of the tubular shaped body 140, from the first end 142 to the opposing second end 146. A slotted opening 154 extends from the opposing second end 146, towards but not fully to, the first end 142. The slotted opening 154 ends at a closed, terminal end or surface 156. The slotted opening 154 is of sufficient size and shaped to allow the stop member 130 to move within (either by moving the outer cannula 114 relative to the multiple dispensing unit housing needle 112, or moving the multiple dispensing unit housing needle 112 relative to the outer cannula 114), but stop (i.e. prevent further movement) when reaching the terminal end or surface 156. This allows the fluid dispensing units 14 to assume either a straight orientation, see FIG. 21, or a pre-bent orientation, see FIG. 16 or 19. The outer cannula 114 is sized and shaped to allow the multiple dispensing unit housing needle 12 to fit and move within. Individual dispensing units such as syringes (not shown) may also be secured to each of the multiple fluid dispensing units 14 as a source of fluid dispensing.

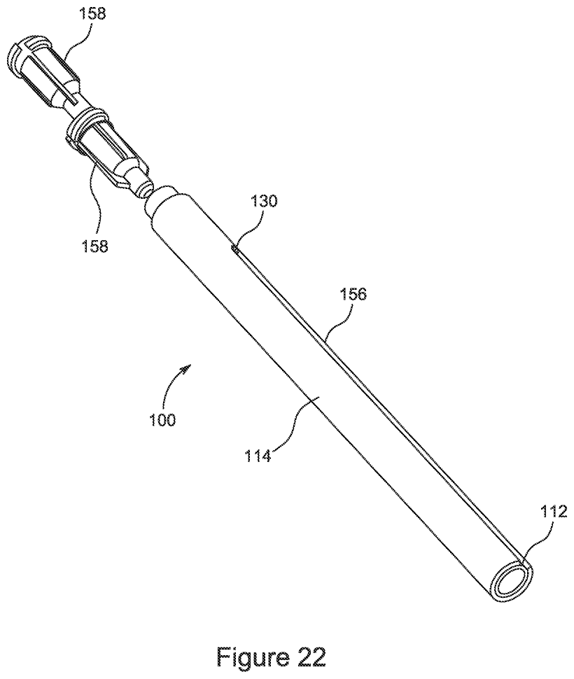

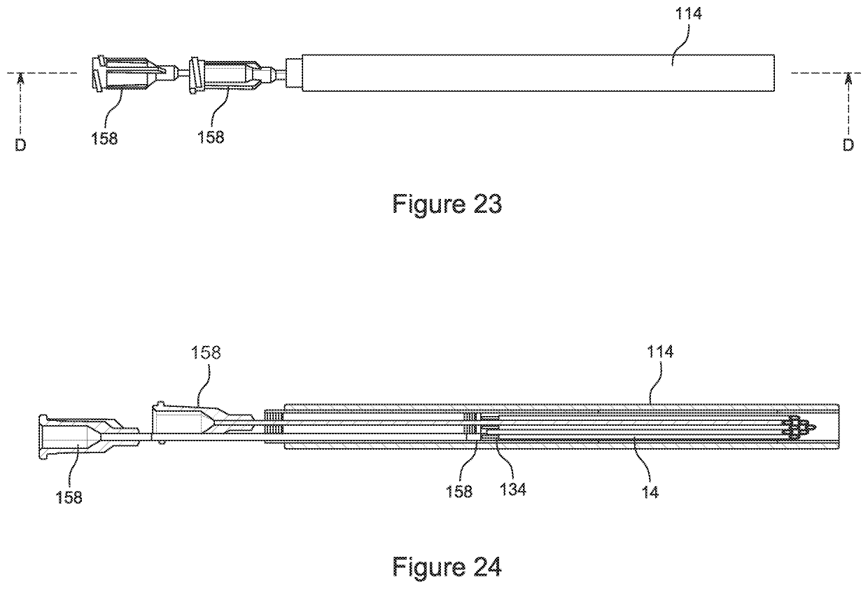

Referring to FIGS. 22-24, the needle having multiple dispensing units 100 is illustrated having a luer lock needle(s). The luer lock needles 158 (or other devices, such as slip tip) are shown engaging to or connecting with a fluid dispensing unit engaging member 134 associated with or connected to an individual fluid dispensing unit 14 (via junction 136). This arrangement allows a user to dispense fluid out of specific, individual fluid dispensing units 14 and/or not dispense fluid out of specific, individual fluid dispensing units 14.

All patents and publications mentioned in this specification are indicative of the levels of those skilled in the art to which the invention pertains.

It is to be understood that while a certain form of the invention is illustrated, it is not to be limited to the specific form or arrangement herein described and shown. It will be apparent to those skilled in the art that various changes may be made without departing from the scope of the invention and the invention is not to be considered limited to what is shown and described in the specification and any drawings/figures included herein.

One skilled in the art will readily appreciate that the present invention is well adapted to carry out the objectives and obtain the ends and advantages mentioned, as well as those inherent therein. The embodiments, methods, procedures and techniques described herein are presently representative of the preferred embodiments, are intended to be exemplary, and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed, various modifications of the described modes for carrying out the invention which are obvious to those skilled in the art are intended to be within the scope of the following claims.

1. A surgical instrument for delivering fluid to multiple areas of an injection site, the surgical instrument comprising:
a housing having a first end and a second end;
a plurality of flexible fluid dispensing units, wherein:
each of the plurality of flexible fluid dispensing units has a first end secured within the housing and a terminal end exposed at the second end of the housing, and
at least one flexible fluid dispensing unit of the plurality of flexible fluid dispensing units has an initial configuration and a secondary configuration that differs from the initial configuration; and
a sleeve slidably engaged around the terminal ends of the plurality of flexible fluid dispensing units, wherein:
the sleeve is slidable from an initial position to a final position, the initial position and the final position relative to the terminal ends of the plurality of flexible fluid dispensing units,
the initial position maintains the at least one flexible fluid dispensing unit in the initial configuration,
the final position permits the at least one flexible fluid dispensing unit to transition to the secondary configuration, and
the at least one flexible fluid dispensing unit in the secondary configuration and the remaining flexible fluid dispensing units in the plurality of flexible fluid dispensing units can deliver the fluid from the first end of the housing to the multiple areas of the injection site when the sleeve is in the final position.

2. The surgical instrument of claim 1, further comprising a barrier that limits an amount of travel of the sleeve relative to the terminal ends of the plurality of flexible fluid dispensing units.

3. The surgical instrument of claim 2, wherein the barrier further comprises a locking ring engaged around an external surface of the housing, and wherein positioning of the locking ring dictates an amount of travel of the terminal ends of the plurality of flexible fluid dispensing units.

4. The surgical instrument of claim 3, wherein an external surface of the housing includes a first mating surface, and wherein the internal surface of the locking ring includes a second mating surface configured to mate with the first mating surface.

5. The surgical instrument of claim 4, wherein the first mating surface is a first threaded interface, and wherein the second mating surface is a second threaded interface configured to engage with the first threaded surface.

6. The surgical instrument of claim 3, wherein an external surface of the locking ring is textured to improve grip. 7 The surgical instrument of claim 1, further comprising an outer cannula, wherein:
the outer cannula has a first end separated from a second end by an elongated body;
the first end is sized to allow the sleeve surrounding the plurality of fluid dispensing units to pass through the first end and into the elongated body, and wherein the outer cannula is configured to prevent at least the sleeve and the housing from exiting from the second end of the outer cannula.

8. The surgical instrument of claim 7, wherein the second end of the outer cannula further comprises a feature that reduces an effective diameter of the second end to secure the sleeve within the elongated body of the outer cannula.

9. The surgical instrument of claim 7, wherein the housing comprises a protuberance extending outwardly from an exterior surface of the housing, and wherein:
the outer cannula further comprises a slotted opening extending from the first end towards the second end, and
the slotted opening is dimensioned to receive the protuberance and prevent the housing from exiting from the second end of the outer cannula.

10. The surgical instrument of claim 9, further comprising a needle cap that includes:
a plurality of tubular bodies for engaging with the plurality of flexible fluid dispensing units;
an attachment interface for engaging with a fluid dispensing device; and
a dimension that allows the needle cap to fit in the outer cannula.

11. The surgical instrument of claim 1, wherein at least some of the plurality of fluid dispensing units differ in a length extending from the second end or angle of curvature.

12. The surgical instrument of claim 1, wherein the plurality of fluid dispensing units are configured for selective delivery of the fluid.

13. The surgical instrument of claim 1, wherein each of the plurality of flexible fluid dispensing units has an initial configuration and a secondary configuration that differs from the first configuration.

14. A method for delivering fluid to multiple areas of an injection site with a surgical instrument, the method comprising:
positioning the surgical instrument at the injection site to deliver the fluid;
advancing a housing towards the injection site, wherein the housing includes a first end and a second end, and wherein a plurality of flexible fluid dispensing units is secured within the housing with terminal ends of the plurality of flexible fluid dispensing units exposed at the second end of the housing;
sliding a sleeve from an initial position to a final position relative the terminal ends of the plurality of flexible fluid dispensing units, wherein the initial position maintains at least one flexible fluid dispensing unit of the plurality of flexible dispensing units in an initial configuration, and wherein the final position permits the at least one flexible fluid dispensing unit to transition to a secondary configuration that differs from the initial configuration;
delivering fluid from the first end of the housing and though the terminal ends of at least some of the plurality of fluid dispensing units to the multiple areas of the injection site.

15. The method of 14, wherein:
positioning the surgical instrument further comprises positioning an outer cannula at the injection site, wherein the outer cannula has a first end separated from a second end by an elongated body, the second end is directed toward the injection site; and
advancing the housing towards the injection site further comprises inserting the housing and the sleeve in the initial position into the elongated housing from the second end.

16. The method of claim 15, wherein advancing the housing further comprises advancing the housing through the elongated body of the outer cannula until a barrier limits advancement through the elongated body of the outer cannula.

17. The method of claim 16, wherein sliding the sleeve from the initial position to the final position further comprises:
causing a distal end of the sleeve to engage a feature that reduces an effective diameter of the second end of the outer cannula; and
causing a proximate end of the sleeve to engage the barrier.

18. The method of claim 15, further comprising:
coupling a barrier to an external surface of the housing based on a desired amount of exposure of the terminal ends of the plurality of fluid dispensing units from the surgical instrument.

19. The method of claim 14, wherein:
positioning the surgical instrument further comprises positioning an outer cannula at the injection site, wherein the outer cannula has a first end separated from a second end by an elongated body defining a slotted opening extending from the first end toward the second end,
advancing the housing towards the injection site from the second end of the outer cannula toward the first end further comprises sliding a protuberance of the housing along a length of the slotted opening, wherein the protuberance extends outwardly from an outer surface of the housing.

20. The method of claim 14, wherein delivering the fluid further comprising delivering fluid from only some of the plurality of flexible fluid dispensing units.
